(19) United States
(12) Patent Application Publication
(10) Pub. No.: US 2025/0380923 A1
(43) Pub. Date: Dec. 18, 2025

(54) METHOD FOR ARTIFACT REDUCTION IN CONE BEAM COMPUTED TOMOGRAPHY IMAGES

(71) Applicant: Milan Madhavji, Mississauga (CA)

(72) Inventor: Milan Madhavji, Mississauga (CA)

(21) Appl. No.: 19/237,598

(22) Filed: Jun. 13, 2025

Related U.S. Application Data
(60) Provisional application No. 63/660,011, filed on Jun. 14, 2024.

(51) Int. Cl. A61B 6/00; A61B 6/51; A61C 9/00

(52) U.S. Cl. CPC A61B 6/5258; A61B 6/51; A61B 6/5247; A61C 9/0053

(57) ABSTRACT

A method includes acquiring a first volumetric image data set representing dentition of a patient, the first volumetric image data set including a modeled structure having an artifact distorting a boundary thereof, aligning a 3D digital impression of dentition of the patient with at least a portion of the first volumetric image data set, segmenting individual structures in the first volumetric image data set, merging the segmented individual structures to form a unitary volumetric model, thickening the 3D digital impression to form a shell bounding the at least a portion of the first volumetric image data set and supplementing the boundary of the at least one modeled structure, and, combining the 3D digital impression and the unitary volumetric model into a second volumetric image data set including the unitary volumetric model having at least a portion thereof bounded by the 3D digital impression.

### CROSS-REFERENCE TO RELATED APPLICATIONS

This application claims the benefit of U.S. Provisional Application No. 63/660,011 filed Jun. 14, 2024, the contents of which are incorporated herein by reference.

### TECHNICAL FIELD

The present invention relates to a method and system for artifact reduction in dental scan data. More particularly, the present invention relates to a method and system for metal artifact reduction in dental scan images via post-processing that utilizes surface data from dental digital impressions.

### BACKGROUND

Cone Beam Computed Tomography (CBCT) is an imaging technique that provides volumetric scans or three-dimensional (3D) images of the teeth, oral and maxillofacial region. CBCT imaging is widely used in dentistry for the diagnosis and treatment planning of various dental conditions. However, one of the challenges in using CBCT imaging is the presence of artifacts, which can cause distortions in the images and compromise image readability. An “artifact” in medical imaging refers to any distortion or error in the image that does not accurately represent the true anatomy or condition of the structure being examined. In the context of CBCT and other imaging modalities, artifacts can interfere with the interpretation of the images, potentially leading to misdiagnosis or inaccurate treatment planning. Artifacts in dental radiology can arise from various sources, including patient movement, technical issues, and the presence of certain materials.

Metal artifacts are a type of noise caused by metal or other radiopaque materials used in dental treatments, such as in dental implants, crowns, posts, and fillings, for example. These materials may cause, among others, beam hardening artifacts, scatter artifacts, and photon starvation artifacts during the imaging process. Metal artifacts usually appear as artificial streaks and dark shadings around image data representing metallic objects in CBCT images and can significantly impair image quality and hinder accurate diagnosis. The presence of metal artifacts in CBCT scans also causes errors in segmentation of teeth.

In dentistry, CBCT images and 3D digital impressions are often aligned for comprehensive treatment planning. This alignment allows for more accurate and effective diagnosis, treatment planning, and execution, particularly in complex cases such as implantology, orthodontics, and reconstructive surgery. However, the presence of metal artifacts in CBCT images results in errors in the alignment of CBCT images with 3D digital impressions.

Various methods have been developed to address the problem of metal artifacts in CBCT imaging. Software-based solutions, including metal artifact reduction (MAR) algorithms and advanced image reconstruction techniques, are known to mitigate the appearance of artifacts. For instance, an iterative reconstruction method involves iteratively refine the image by comparing it with a model, thereby reducing artifacts. Another known solution involves dual energy-based methods which aim to reduce the beam hardening artifact by acquiring the projection data at two different X-ray voltage settings to estimate the mono-energetic projection data. However, this method may result in an increase in the X-ray dose to the patient. Pre-scan strategies, like removing removable metal objects and instructing patients on staying still, can reduce artifact impact. Additionally, deep learning-based methods have also been proposed to reduce the metal artifacts directly from the CBCT image. However, deep learning methods require an enormous dataset of patient images to effectively train the deep learning network, thereby limiting the use of this technique.

The known methods for minimizing or eliminating metal artifacts in CBCT imaging have limitations and are not always effective. There is a need for a reliable and effective method for eliminating metal artifacts from CBCT scans.

### BRIEF SUMMARY

The present invention relates to a method and system for artifact reduction in dental scan data. More particularly, the present invention relates to a method and system for metal artifact reduction in dental scan images via post-processing that utilizes surface data from dental digital impressions.

In one aspect, there is provided a method including the steps of: acquiring a first volumetric image data set representing dentition of a patient, the first volumetric image data set including a modeled structure having an artifact distorting a boundary thereof; aligning a 3D digital impression of dentition of the patient with at least a portion of the first volumetric image data set; segmenting individual structures in the first volumetric image data set; merging the segmented individual structures to form a unitary volumetric model; thickening the 3D digital impression to form a shell bounding the at least a portion of the first volumetric image data set and supplementing the boundary of the at least one modeled structure; and, combining the 3D digital impression and the unitary volumetric model into a second volumetric image data set including the unitary volumetric model having at least a portion thereof bounded by the 3D digital impression. The method may further include the step of rendering the second volumetric image data set to provide a volumetric model having realistic appearance. The 3D digital impression may be generated using an intra-oral optical scanner. In one aspect, thickening the 3D digital impression includes further thickening representations of tooth crowns of the 3D digital impression.

In one aspect, the artifact includes a streak extending beyond the boundary and the method further comprises removing the streak. In another aspect, the artifact includes a dark region and dark region is bounded by the shell. In one aspect, the shell has a uniform thickness.

In one aspect, segmenting further includes segmenting each of a modeled maxilla and modeled mandible of the first volumetric image data set, providing decoupled volumetric image data representing the modeled maxilla, and providing decoupled volumetric image data for the modeled mandible. The method may further include spacing apart the decoupled volumetric image data representing the modeled maxilla and the decoupled volumetric image data representing the modeled mandible. The 3D digital impression may be aligned with one of the modeled mandible and the modeled maxilla.

In another aspect, there is provided a system including a capture module configured to acquire a first volumetric image data set representing dentition of a patient, the first volumetric image data set including a modeled structure having an artifact distorting a boundary thereof, an alignment module configured to align a 3D digital impression of dentition of the patient with at least a portion of the first volumetric image data set, a segmentation module configured to segment individual structures in the first volumetric image data set, a modeling module configured to merge the segmented individual structures to form a unitary volumetric model, an impression module configured to thicken the 3D digital impression to form a shell bounding the at least a portion of the first volumetric image data set and supplementing the boundary of the at least one modeled structure, and wherein the modeling module is further configured to combine the 3D digital impression and the unitary volumetric model into a second volumetric image data set including the unitary volumetric model having at least a portion thereof bounded by the 3D digital impression. The 3D digital impression is generated using an intra-oral optical scanner.

The impression module may be further configured to further thicken representations of tooth crowns of the 3D digital impression. In one aspect, the impression module is configured to thicken the shell to a uniform thickness.

The system may further include a rendering module configured to render the second volumetric image data set to provide a volumetric model having realistic appearance.

In one aspect, the artifact includes a streak extending beyond the boundary and the modeling module is further configured to remove the streak. In another aspect, the artifact includes a dark region and the modeling module is configured to bound the dark region within the shell.

In one aspect, the segmentation module is further configured to segment each of a modeled maxilla and modeled mandible of the first volumetric image data set, provide decoupled volumetric image data representing the modeled maxilla and provide decoupled volumetric image data for the modeled mandible. In another aspect, the segmentation module is further configured to space apart the decoupled volumetric image data representing the modeled maxilla and the decoupled volumetric image data representing the modeled mandible. The alignment module may be configured to align the 3D digital impression to one of the modeled mandible and the modeled maxilla.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system for decoupling dental arches in dental scan data in accordance with one aspect;

FIG. 2 illustrates a method for artifact reduction in accordance with one aspect;

FIG. 3 illustrates a method for artifact reduction in accordance with one aspect;

FIG. 4 illustrates an image processing module for artifact reduction in accordance with one aspect;

FIG. 5 illustrates a first volumetric image data set in accordance with one aspect;

FIG. 6 illustrates a first volumetric image data set having a 3D digital impression aligned therewith in accordance with one aspect;

FIG. 7 illustrates the first volumetric image data set of FIG. 6 post-segmentation in accordance with one aspect;

FIG. 8 illustrates a unitary volumetric model in accordance with one aspect;

FIG. 9 illustrates a unitary volumetric model in accordance with one aspect;

FIG. 10 illustrates a shell formed around the unitary volumetric model of FIG. 9 in accordance with one aspect;

FIG. 11 illustrates a second volumetric image data set in accordance with one aspect; and,

FIG. 12 illustrates a second volumetric image data set in accordance with one aspect.

### DETAILED DESCRIPTION

The present invention relates to a method and system for artifact reduction in dental scan data. More particularly, the present invention relates to a method and system for metal artifact reduction in dental scan images via post-processing that utilizes surface data from dental digital impressions.

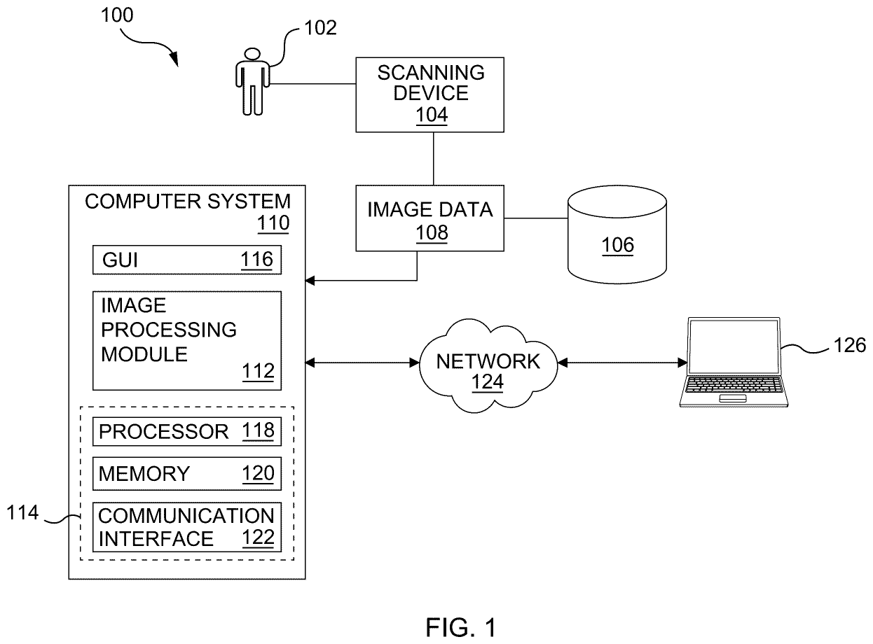

FIG. 1 illustrates a system 100 for artifact reduction in a volumetric model of dentition of a patient, in accordance with one aspect.

System 100 includes computer system 110 for analyzing image data 108 representing dentition of a patient 102. Image data 108 is acquired using a scanning device 104 which may then be provided directly to computer system 110 or which may be retrieved by computer system 110 from data storage 106. Scanning device 104 may be any suitable scanning device such as intraoral scanners, cone beam computed tomography (CBCT) scanners, x-ray machines, and the like.

Image data 108 is preferably three-dimensional image data 108 and in a format of or capable of being converted into a volumetric model including volumetric representations of the various dental structures of the patient 102, including the upper and lower jawbones, and surrounding tissues of the patient 102. In the context of a three-dimensional model, such representations may be referred to as “modeled structures”. In one aspect, image data 108 is acquired in Digital Imaging and Communications in Medicine (DICOM) format. DICOM is a universal standard for the storage, handling, and transmission of medical images and associated information. It ensures compatibility and interoperability among different systems and devices by standardizing the format and including comprehensive metadata such as patient identification, image type, and device information. DICOM files are integral in maintaining the integrity and consistency of medical data as they include both the image and its complete context-information critical to accurate diagnosis and treatment planning.

Computer system 110 includes a controller 114, a graphical user interface (GUI) 116, and an image processing module 112. The controller 114 includes at least one processor 118, a memory 120 configured to store one or more first program instructions for execution by system 100 and at least one communication interface 122.

The processor 118 may include one or more processing elements, micro-controllers, circuitry, field programmable gate array (FPGA) or other processing system, and resident or external memory for storing data, executable code, and other information accessed or generated by the computer system 110. Therefore, processor 118 may include any microprocessor device configured to execute algorithms or program instructions. In general, the term “processor”, may be broadly defined to encompass any device having one or more processing elements, which execute a set of program instructions from one or more processing elements and/or which execute a set of program instructions from a non-transitory memory medium, where the set of program instructions is configured to cause the one or more processors to carry out any of the one or more process steps.

The memory 120 may include any storage medium known in the art suitable for storing the set of program instructions executable by the associated one or more processors. For example, memory 120 may include a non-transitory memory medium. Memory 120 may include but is not limited to, a read-only memory (ROM), a random access memory (RAM), a magnetic or optical memory device (e.g., disk), a magnetic tape, a solid state drive, flash memory (e.g., a secure digital (SD) memory card, a mini-SD memory card, and/or a micro-SD memory card), universal serial bus (USB) memory devices, and the like. The memory 120 may be housed in a common controller housing with the one or more processors. Alternatively or in addition, the memory 120 may be located remotely with respect to the spatial location of the processors and/or the controller 114 may access a remote memory (e.g., server), accessible through a network (e.g., internet, intranet, and the like).

The controller 114 may be configured to perform one or more process steps, as defined by the one or more sets of program instructions. The one or more process steps may be performed iteratively, concurrently and/or sequentially. The one or more sets of program instructions may be configured to operate via a control algorithm, a neural network (e.g., with states represented as nodes and hidden nodes and transitioning between them until an output is reached via branch metrics), a kernel-based classification method, a Support Vector Machine (SVM) approach, canonical-correlation analysis (CCA), factor analysis, flexible discriminant analysis (FDA), principal component analysis (PCA), multidimensional scaling (MDS), principal component regression (PCR), projection pursuit, data mining, prediction-making, exploratory data analysis, supervised learning analysis, Boolean logic (e.g., resulting in an output of a complete truth or complete false value), fuzzy logic (e.g., resulting in an output of one or more partial truth values instead of a complete truth or complete false value), or the like. For example, in the case of a control algorithm, the one or more sets of program instructions may be configured to operate via proportional control, feedback control, feedforward control, integral control, proportional-derivative (PD) control, proportional-integral-derivative (PID) control, or the like.

The communication interface 122 may be operatively configured to communicate with one or more components of the computer system 110 and/or controller 114. For example, communication interface 122 may also be coupled (e.g., physically, electronically, and/or communicatively) with the at least one processor 118 to facilitate data transfer between components of the computer system 110, other components of system 100 and processor 118. For instance, the communication interface 122 may be configured to retrieve data from the at least one processor 118, or other devices, transmit data for storage in the memory 120, retrieve data from storage in the memory 120, or the like. By way of another example, controller 114 may be configured to receive and/or acquire data or information from other systems or tools by a transmission medium that may include wireline and/or wireless portions. By way of another example, controller 114 may be configured to transmit data or information (e.g., the output of one or more procedures of the inventive aspects disclosed herein) to one or more systems or tools by a transmission medium that may include wireline and/or wireless portions (e.g., a transmitter, receiver, transceiver, physical connection interface or any combination thereof). In this regard, the transmission medium may serve as a data link between the controller 114 and the other components of the computer system 110 and system 100. In addition, controller 114 may be configured to send data to external systems via a transmission medium (e.g., network connection).

In general, the word “module” as used herein, refers to a collection of hardware components and/or software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software may be stored, partially or fully, on a memory device of the executing computing device, for execution by the computing device. Software instructions may be embedded in firmware, such as an EPROM. It will be further appreciated that hardware devices (such as processors and CPUs) may be comprised of connected logic units, such as gates and flip-flops, and/or may be comprised of programmable units, such as programmable gate arrays or processors. The modules or computing device functionality described herein are preferably implemented as software modules but may be represented in hardware devices. Generally, the modules described herein refer to hardware or software modules that may be combined with other modules or divided into sub-modules despite their physical organization or storage.

Embedded within or accessible to the computer system 110 is image processing module 112. “Image processing module” refers to one or more computer components, which may include hardware or software, which are designed to collect, create, edit, process, analyze, and display image data. Such components may be local to the image processing module or external and in data exchange communication therewith. Image processing module is preferably medical image processing module configured to manage and process images obtained from various diagnostic tools such as X-rays, CT scans, MRIs, ultrasound, and other imaging modalities. Image processing module 112 can handle various image formats from simple photographs to complex graphics and medical scans. Image processing module 112 may exist in various forms, such as being embedded on a hard drive of computer system 110, stored on a server in data communication with computer system 110 or is accessible as a third-party software that can be used as a service by computer system 110. In another aspect, image processing module 112 may include one or more machine learning models or an “artificial intelligence” system capable of performing automated image analysis, accessible by computer system 110. Image processing module 112 is configured to receive as input image data 108 acquired from the scanning device 104 or images stored in data storage 106 or elsewhere that is accessible by image processing module 112. Image processing module 112 may acquire the image data 108 automatically as a function of system 100 or may be instructed to acquire image data 108 by user input via graphical user interface 116, with graphical user interface 116 being in data exchange communication with image processing module 112. Once image data 108 is acquired by computer system 110 and is accessible to image processing module 112, image processing module 112 is configured to delineate various modeled structures of the patient's dentition and/or mask various structures, and selectively enable relative movement between modeled jaw or arch structures and to output a second image data set which, in some aspects, may be a digitally altered, reconstructed or modified image data set. Image processing module 112 can enhance the visibility of anatomical structures, improve image quality by digitally decoupling or separating the modeled upper and lower jaws to eliminate overlapping of modeled teeth. Use of imaging software can lead to a more accurate and efficient way to analyze 3D or CBCT scan data, improving the overall quality of dental care.

The computer system 110 is in data exchange communication with a user device 126 via network 124. The network 124 may comprise any suitable network or networks, including a local area network (LAN), wide area network (WAN), Internet, or combination thereof. For example, the network 124 may include a wireless cellular service (e.g., 4G). Generally, the network 124 enables bidirectional communication between the computer system 110 and the user device 126. In some aspects, network 124 may comprise a cellular base station, such as cell tower(s), communicating to the one or more components of the system 100 via wired/wireless communications based on any one or more of various mobile phone standards, including NMT, GSM, CDMA, UMMTS, LTE, 5G, or the like. Additionally or alternatively, network 124 may comprise one or more routers, wireless switches, or other such wireless connection points communicating to the components of the computer system 110 via wireless communications based on any one or more of various wireless standards, including by non-limiting example, IEEE 802.11a/b/c/g (WIFI), the BLUETOOTH standard, or the like.

User device 126 may be any suitable device, for example, a laptop, a computer, a smart phone, a tablet, and the like. User device 126 is operable by a healthcare practitioner to access the digitally adjusted images of the patient 102 created using the image processing module 112 of the computer system 110.

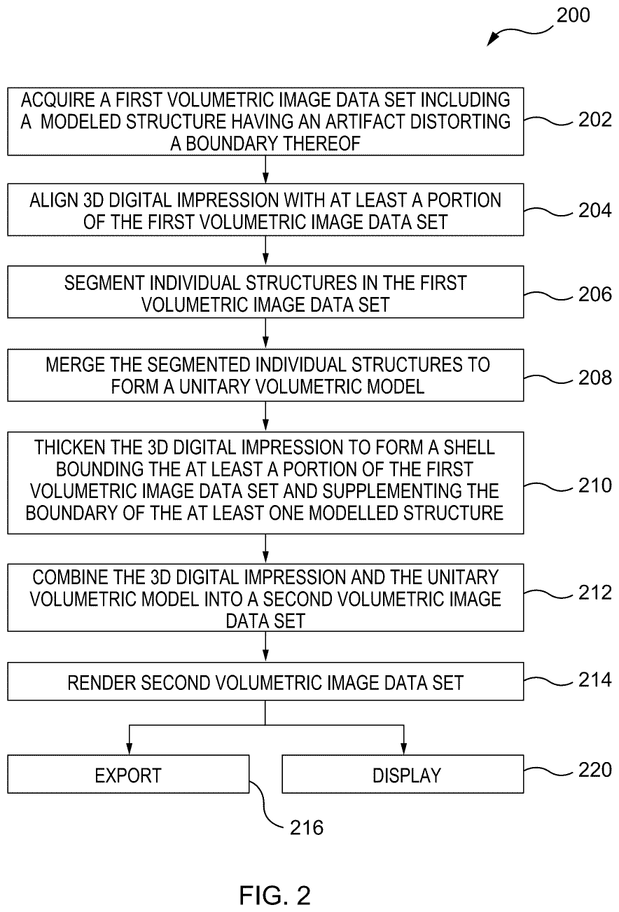

FIG. 2 illustrates a method 200 for reducing or minimizing artifacts in image data representing a volumetric model of a patient's dentition, according to one aspect. Although the example method 200 depicts a particular sequence of operations, the sequence may be altered without departing from the scope of the present disclosure. For example, some of the operations depicted may be performed in parallel or in a different sequence that does not materially affect the function of the method 200.

Image artifacts are a common issue in medical imaging. One common source for image artifacts is the presence of metallic objects such as dental appliances, fillings, crowns or implants which, when scanned in an x-ray scanning operation cause x-rays to reflect, scatter or fail to penetrate materials as expected. Image artifacts or metal artifacts can include beam hardening, scatter radiation, and photon starvation, among others. Beam hardening occurs when lower-energy X-rays are absorbed by the metal, leaving higher-energy X-rays to pass through. This differential absorption can create streak artifacts that obscure adjacent structures and result in dark bands in the image. Scatter radiation refers to scattering of X-ray photons in multiple directions by the metal objects, resulting in noise and reduced image contrast. Photon starvation is caused by absorption of X-ray photons by metallic objects, resulting in areas with insufficient data and creating shadows in the image. Therefore, image artifacts or metal artifacts can degrade image quality and hinder accurate diagnosis and treatment planning. The method 200 of FIG. 2 provides at least one technique for reducing or eliminating metal artifacts in image data via post-processing using other data, such as a 3D digital impression of the patient's dentition. 3D digital impressions are typically taken by optical scanning devices and are therefore not subject to x-ray imaging artifacts.

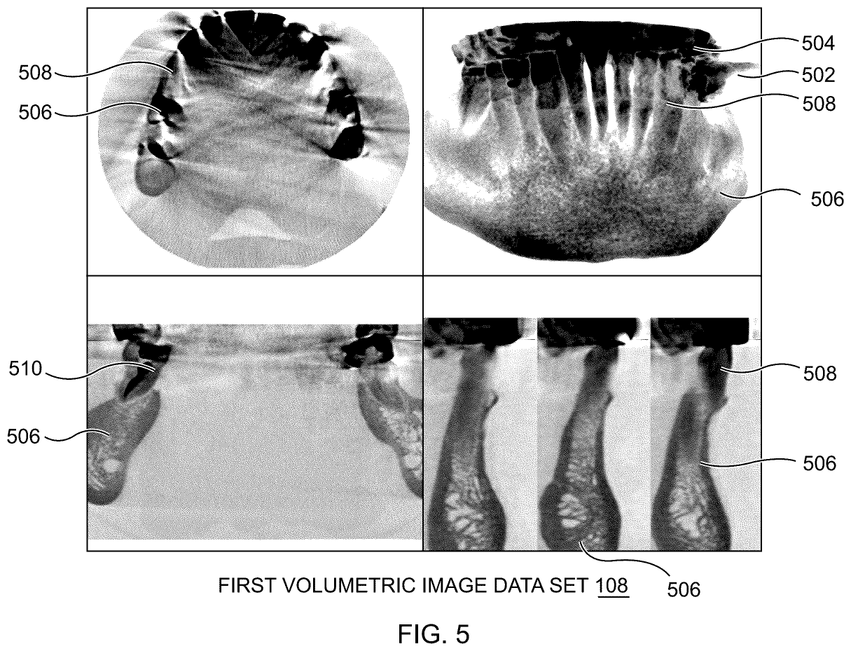

In block 202, method 200 acquires a first volumetric image data set representing dentition of a patient. In one aspect, the first volumetric image data set is the image data 108 of FIG. 1 and includes a modeled structure having an artifact distorting a boundary thereof. The first volumetric image data set is a volumetric image data set which includes modeled dental structures of a patient's dentition such as modeled maxilla 504, modeled mandible 506 (FIG. 5), as well as modeled soft tissues, nerve paths, and other bones in the craniofacial region. The first volumetric image data set may be obtained using a suitable x-ray imaging modality, such as via one or more CBCT scanning operations. The first volumetric image data set provides a comprehensive model that is useful for accurate diagnosis and treatment planning in various dental and medical fields.

An example CBCT scan data of a patient's dentition is shown in FIG. 5. As shown in FIG. 5, the CBCT scan data in the closed bite position may include some overlap between the modeled structures, such as the modeled maxillary and mandibular teeth. Areas of overlap may include, for example, interdigitating surfaces of modeled teeth or molars between the modeled upper arch and modeled lower arch.

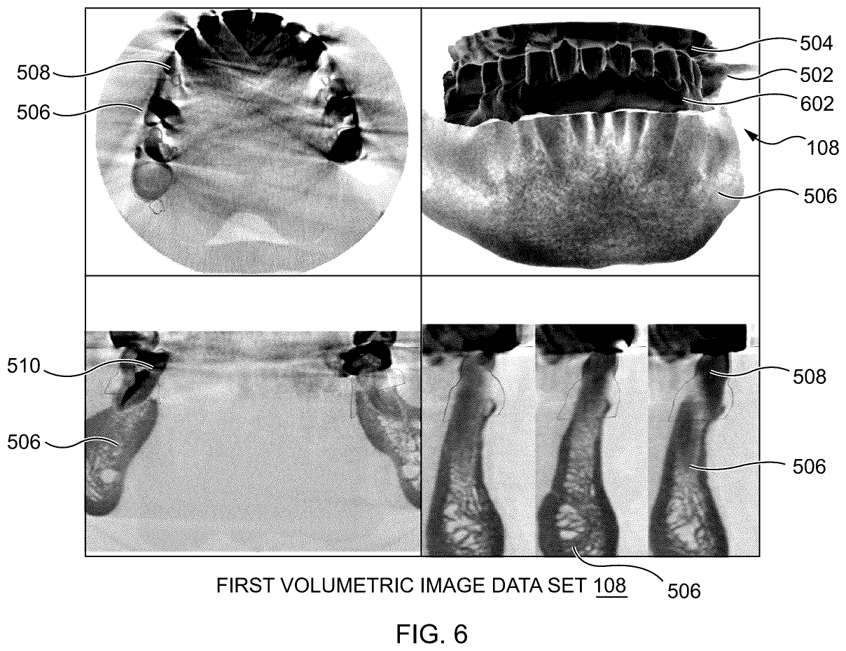

In block 204, method 200 aligns a 3D digital impression 602 (FIG. 6) of dentition of the patient with at least a portion of the first volumetric image data set. A 3D digital impression captures the surface geometry of the patient's dentition and creates a detailed digital model of the teeth and gums. In one aspect, the 3D digital impression 602 is obtained using an intra-oral optical scanner. This is advantageous because image data obtained by means of optical scanning is not subject to the artifacts which affect x-ray imaging modalities. Therefore, such data can be used as described hereinafter to supplement structural boundaries and correct image artifacts.

Alignment may include superimposing the 3D digital impression 602 over the first volumetric image data set and either manually or automatically aligning the 3D digital impression 602 with the first volumetric image data set. The alignment may involve identifying common anatomical landmarks such as the cusp tips, occlusal surfaces, or remaining unaffected regions of the teeth. The alignment may be performed either manually by user or using automatic alignment tools provided by dental imaging software. If significant artifacts are present in the first volumetric image data set, accurate alignment may be difficult to achieve with an automated alignment tool due to the absence of adequate definitive registration points in the two datasets. In such cases, an automated alignment of the 3D digital impression with the first volumetric image data set can be manually verified by a user to correct any discrepancies in the alignment. In one aspect, once proper alignment is achieved, the aligned 3D digital impression and first volumetric image data set together accurately reflect both the internal structures from the first volumetric image data set and the surface details from the 3D digital impression.

In block 206, method 200 segments individual structures in the first volumetric image data set. Segmentation refers to the process of isolating and distinguishing distinct structures and sub-structures in the first volumetric image data set and can be carried out using known techniques. Various specialized software tools are available that can perform dental segmentation, often utilizing artificial intelligence (AI) to enhance accuracy and efficiency. In some aspects, once the segmentation is complete, the data, which originally is in voxel format, may be used to generate a mesh. This segmented mesh is a collection of vertices, edges, and faces that approximate the shape of each original structure or teeth in 3D space.

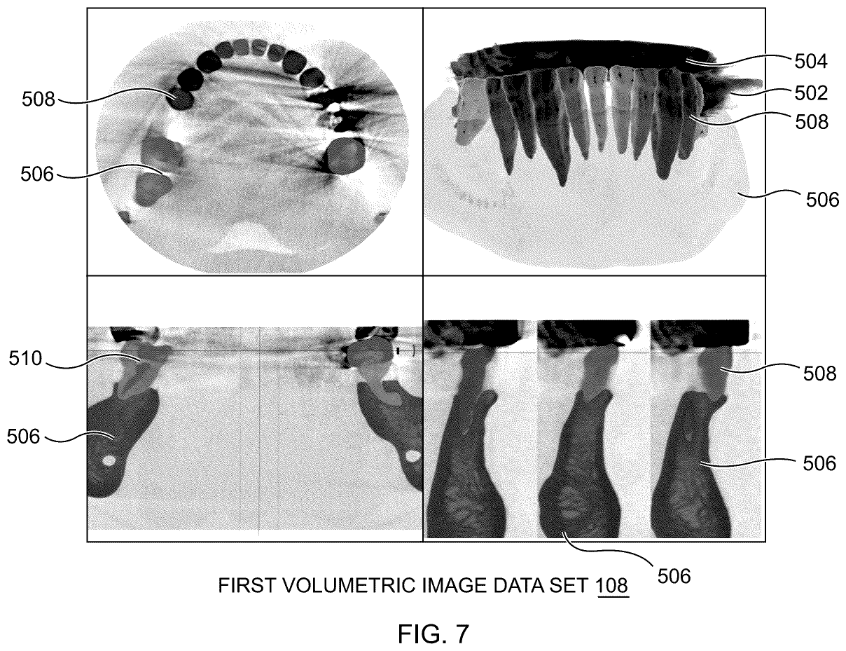

As described in further detail hereinafter, FIG. 7 shows the first volumetric image data set, representing patient dentition of FIG. 5, with individual modeled structures, including those of the modeled upper arch and modeled lower arch segmented using suitable segmentation techniques. Segmentation refers to the technique of delineating and separating overlapping anatomical regions within an image data set.

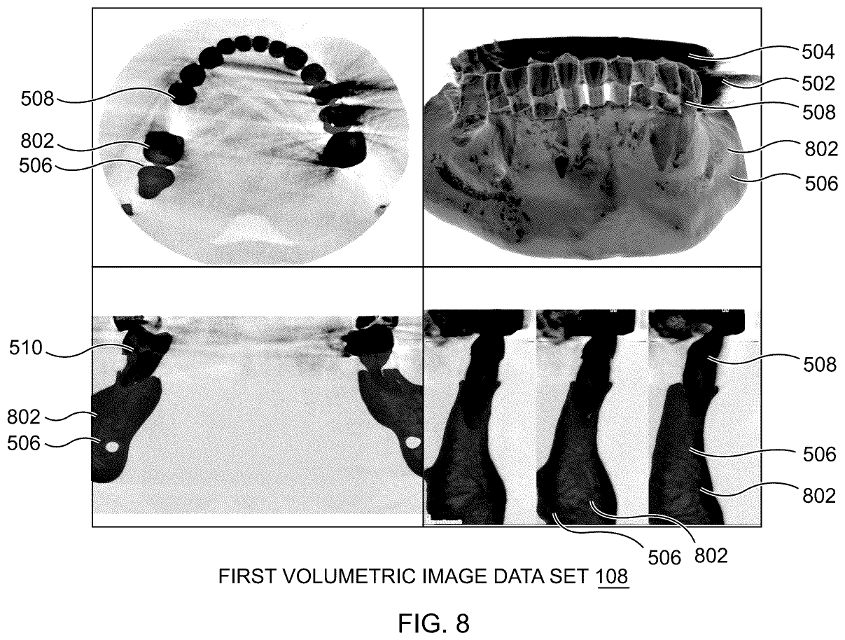

In block 208, method 200 merges the segmented individual structures to form a unitary volumetric model 802 (FIG. 8). In one aspect, the unitary volumetric model 802 is formed by merging the segmented individual structures in a region of interest in the first volumetric image data set. One limitation posed by image artifacts, particularly those posed by metal materials, is that in addition to producing streaks emanating from the modeled tooth surface, they may also misrepresent the internal opacity of the modeled structure. This results in portions of the dentition in the first volumetric image data set appearing as dark grey or black, or in some cases bright white, thereby giving the inaccurate impression that the tooth surface is absent in some regions of the unitary volumetric model 802.

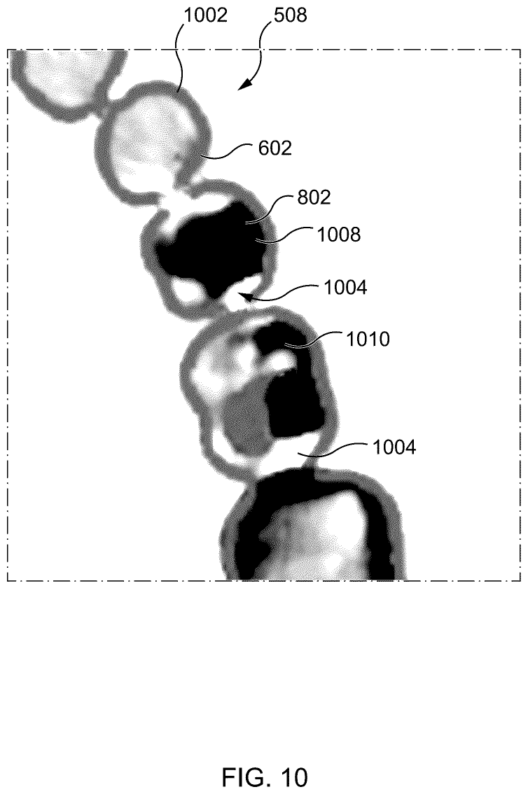

In block 210, method 200 thickens the 3D digital impression to form a shell 1002 (FIG. 10) bounding the at least a portion of the first volumetric image data set and supplementing the boundary of the at least one modeled structure. The shell completes or enhances the boundary of the at least one modeled structure both serving to replace or substitute the boundary in the event of a streak or repairing it in the event of a deleterious artifact such as a beam hardening artifact. The thickened shell 1002 reconstructs or repairs the modeled tooth surfaces that are distorted by the artifact. Such surfaces may be streaked or may appear to be absent in the unitary volumetric model, as shown in FIG. 8. In one aspect, the shell may overwrite or replace the boundary of the at least one modeled structure. In one aspect, the thickness of the shell 1002 is set to a uniform thickness, such as 0.5 mm. In some aspects, the gingiva may be deleted or masked from the unitary volumetric model 802 in order to provide a clearer image of the modeled teeth and bones. The shell position is maintained to replicate the tooth shape and position in the aligned 3D digital impression. The 3D digital impression may be colored to provide a first volumetric image data set having realistic coloration or appearance. In another aspect, white (or light grey) color of the shell 1002 is applied to the first volumetric image data set, in order to generate a well-defined modeled tooth surface therein.

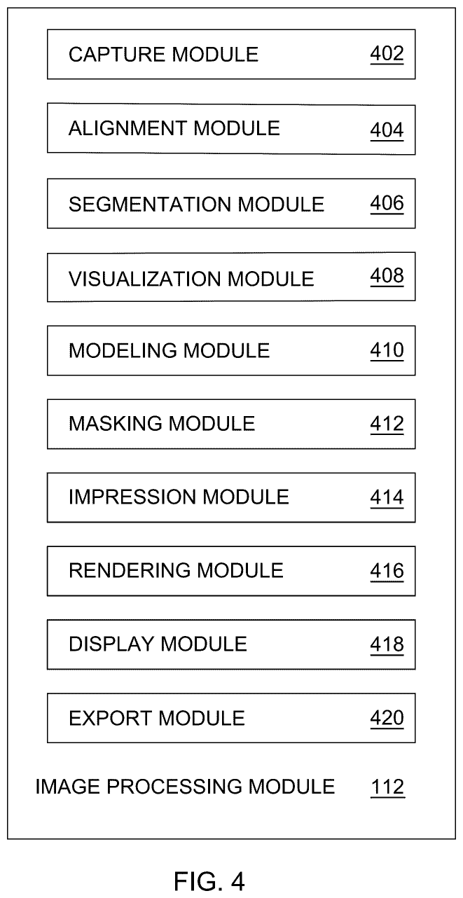

In block 212, method 200 combines the 3D digital impression 602 and the unitary volumetric model 802 into a second volumetric image data set 1100 including the unitary volumetric model 802 having at least a portion thereof bounded by the 3D digital impression 602. In one aspect, the first volumetric image data set and 3D digital impression 602 may be imported into a modeling module 410 (FIG. 4) in data exchange communication with or having embedded thereon specialized dental imaging software which facilitates combination of the two image data sets, by automatic, semi-automatic or manual means.

In one optional aspect, method 200 may include, in block 214, rendering the second volumetric image data set 1100 into a volumetric model having a realistic visual presentation. This may include coloration of the second volumetric image data set 1100 in a manner similar to the color of the patient's dentition or other post-processing techniques to make the second volumetric image data set 1100 more life-like or more suitable for treatment planning. This may be accomplished, in one aspect, via rendering module 416.

In one aspect, the method 200 may further include exporting the digitally created scan to a DICOM format, as shown in block 216. DICOM is the standard format for handling, storing, and transmitting information in medical imaging. Exporting the digitally simulated scan into DICOM format allows the data to be easily shared and accessed across different medical imaging systems and platforms used by various healthcare professionals. The exported DICOM files of segmented teeth can be utilized in various dental software tools for further analysis, treatment planning, and even for the creation of orthodontic appliances or surgical guides.

In one aspect, the method 200 further includes displaying the second volumetric image data set 1100, as shown in block 220. The second volumetric image data set 1100 illustrates a well-defined and accurate representation of the patient's dentition provided by the first volumetric image data set, with surface morphology that would match the 3D digital impression 602. With the integrated data provided by the second volumetric image data set 1100, dentists can plan treatments more accurately. For example, the combined data helps in creating surgical guides, and ensuring precise placement of implants. It also helps in designing custom dental appliances that fit perfectly with the patient's unique anatomy.

The first volumetric image data set may be taken in a closed bite position or an open bite position, depending on patient and/or imaging technician preference. Image data is generally captured as a unitary volume of pixels or voxels and it can be advantageous, in some aspects, to decouple modeled components from one another as this can provide access to new visualizations of the volumetric image data. Decoupling of modeled components typically becomes available with segmentation as this is when individual modeled components of the unitary image volume are identified and labeled. In particular, it is advantageous to obtain volumetric image data wherein the modeled maxilla 504 and modeled mandible 506 are decoupled from one another and preferably are manipulable as independent components in the unitary volumetric model obtained post-segmentation.

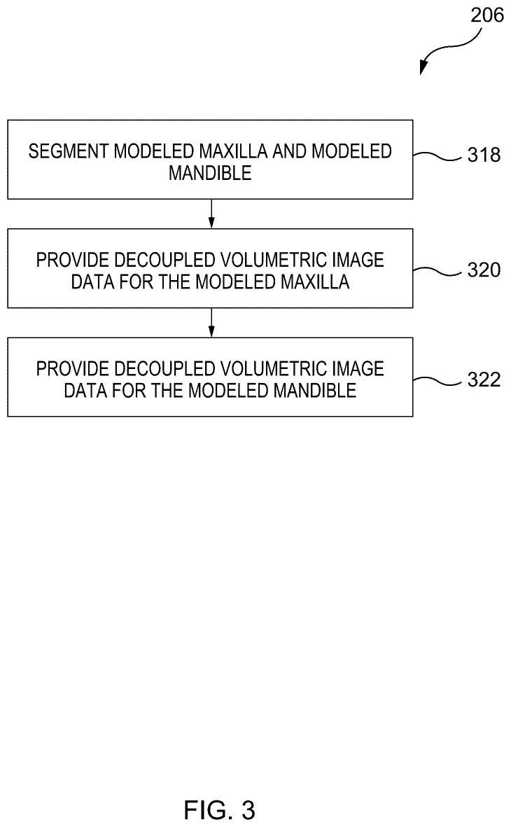

In the aspect wherein the modeled maxilla 504 and modeled mandible 506 are decoupled from one another in the unitary volumetric model, the segmentation shown at block 206 further includes the series of steps shown in FIG. 3.

At block 318, the modeled maxilla and modeled mandible 506 are segmented in the first volumetric image data set. At block 320, the modeled mandible 506 arch is masked or removed from the segmented first volumetric image data set to provide decoupled volumetric image data for the modeled maxilla 504. At block 322, the modeled maxilla 504 is masked or removed from the segmented first volumetric image data set to provide decoupled volumetric image data for the modeled mandible 506.

The method may then resume at block 208 wherein method 200 merges the segmented individual structures to form a unitary volumetric model. Thereby, there is provided a unitary volumetric model having the modeled maxilla 504 and the modeled mandible 506 decoupled from one another.

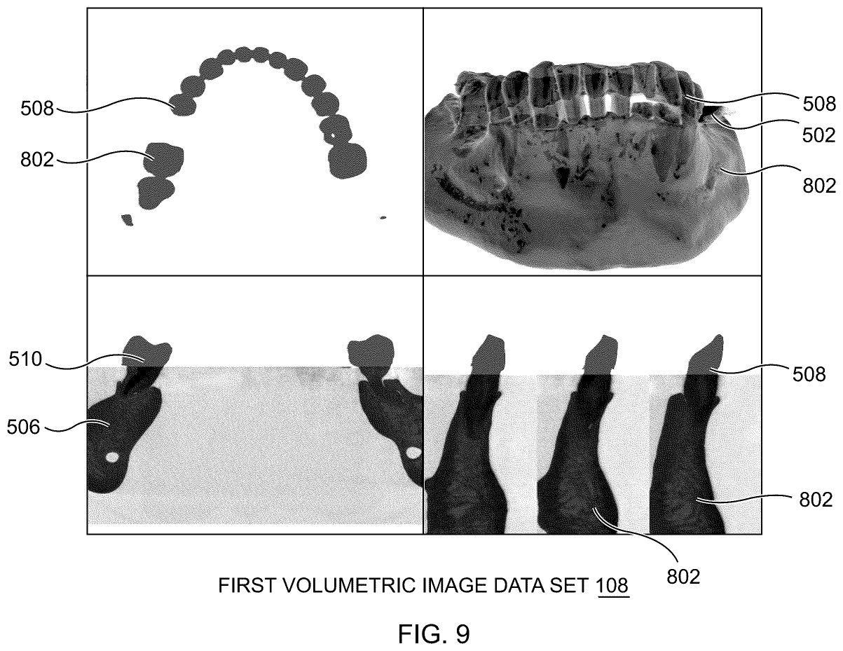

According to some examples, FIG. 8 illustrates merging of the segmented structures in the modeled mandible 506 into the unitary volumetric model. FIG. 9 illustrates an isolated unitary volumetric model of the lower arch or mandible with the remaining portions, such as the upper arch, digitally masked or removed.

In one aspect, some portions of the modeled maxilla 504 may be retained adjacent to the modeled mandibular teeth to include a broad zone around the crowns of the modeled mandibular teeth. The broad zone is included to clean up metal artifact scatter and streaking around the crowns of the mandibular teeth prior to combining the 3D digital impression and the unitary volumetric model into a second volumetric image data set. The metal artifacts can, in addition to producing streaks emanating from the tooth itself, change the internal opacity of the structure, sometimes making teeth appear dark grey or black in regions. As a result, the tooth surface may appear to be absent in some regions, as can be seen in FIG. 9.

FIG. 4 illustrates an image processing module 112 for reducing or minimizing artifacts in image data representing a volumetric model of a patient's dentition, according to one aspect.

Image processing module 112 includes a capture module 402 configured to acquire the first volumetric image data set representing dentition of the patient. As described with respect to FIG. 2, the first volumetric image data set includes a modeled structure having an artifact distorting a boundary thereof.

Image processing module 112 further includes an alignment module 404 configured to align a 3D digital impression of dentition of the patient with at least a portion of the first volumetric image data set. In one aspect, 3D digital impressions is obtained by use of an intra-oral optical scanning device. An intra-oral scanning process involves the use of an optical scanning device, typically a handheld intra-oral scanner, to capture a 3D digital impression of a patient's intra-oral cavity. During the procedure, the scanner emits a light source, often structured light or laser, that reflects off the surfaces of the teeth and soft tissues. These reflected light patterns are detected by sensors within the device and are processed to create a detailed digital representation of the surfaces of a patient's oral anatomy.

Image processing module 112 includes segmentation module 406 configured to segment the individual structures in the first volumetric image data set received from capture module 402. Segmentation module 406 utilizes advanced algorithms to automatically or semi-automatically distinguish and isolate different anatomical structures within the dental scans, such as teeth, bones, nerves, and soft tissues. By accurately segmenting these structures, segmentation module 406 provides dentists and oral surgeons with precise, detailed visualizations that aid in diagnosis, treatment planning, and surgical simulation. The segmentation module 406 may include a user-friendly interface that allows dental professionals to refine and adjust the segmentation to accommodate individual anatomical variations, thereby enhancing the accuracy and effectiveness of dental treatments. Additionally, the integration of machine learning and artificial intelligence in segmentation can further improve the speed and accuracy of the segmentation process.

In the aspect of FIG. 3, wherein the modeled maxilla 504 and modeled mandible 506 are decoupled from one another in the unitary volumetric model, the segmentation module 406 is further configured to segment the modeled maxilla 504 and modeled mandible 506 in the first volumetric image data set. The modeled mandible 506 is masked or removed from the segmented first volumetric image data set to provide decoupled volumetric image data for the modeled maxilla 504 and the modeled maxilla is masked or removed from the segmented first volumetric image data set to provide decoupled volumetric image data for the modeled mandible 506. The segmentation module 406 is further configured to merge the segmented individual structures to form a unitary volumetric model. Thereby, there is provided a unitary volumetric model having the modeled maxilla 504 and the modeled mandible 506 decoupled from one another.

Image processing module 112 includes visualization module 408, which provides tools for customizing the visual representation of the first volumetric image data set segmented by the segmentation module 406. Visualization module 408 may include options for visual enhancement, such as adjusting transparency, color intensity, and applying different color maps to various tissue for more detailed analysis. Visualization module 408 enhances the visualization of the segmented structures. Visualization module 408 may automatically assign different colors for different anatomical structures like teeth, bones, nerves, and soft tissues, segmented by the segmentation module 406. This color differentiation helps in easily distinguishing these structures visually, aiding in better assessment and planning. For example, different types of teeth may be highlighted in different colors within the segmentation view as illustrated in FIG. 7. Visualization module 408 may be an embedded or integrated component of the segmentation module 406 or may be separate from the segmentation module 406 and in data exchange communication therewith.

In one aspect, visualization module 408 further includes a range of tools and/or algorithms that allow dental professionals to adjust image parameters such as brightness, contrast, and sharpness, as well as apply advanced processing techniques like noise reduction, edge enhancement, and geometric transformations like rotation and scaling. Such tools may also include specialized functions such as panoramic reconstruction and the ability to filter specific frequencies to highlight particular structures, such as soft tissues or dense bony areas.

Image processing module 112 further includes a modeling module 410 configured to merge or combine the segmented individual structures to form a unitary volumetric model.

Image processing module 112 further includes a masking module 412 configured to mask a one of the modeled maxilla 504 and the modeled mandible 506 to obtain decoupled volumetric image data for one or both of the modeled maxilla 504 and the modeled mandible 506.

Image processing module 112 further includes impression module 414 which is configured to adjust the thickness of the 3D digital impression, and preferably thickening the 3D digital impression, to form a shell bounding the at least a portion of the first volumetric image data set and supplementing the boundary of the at least one modeled structure. Impression module 414 may include inputs for such adjustments. Such inputs may include user-input controls such as “drag and drop” controls or directional inputs which may be interacted with by a user to increase or reduce thickness of the 3D digital impression.

Modeling module 410 is further configured to combine the 3D digital impression and the unitary volumetric model into a second volumetric image data set including the unitary volumetric model having at least a portion thereof bounded by the 3D digital impression.

Image processing module 112 further includes rendering module 416 which is configured to render the second volumetric image data set 1100 into a volumetric model having a realistic visual presentation. This is advantageous for treatment planning as it provides a realistically-colored digital model of the patient's dentition for use by clinicians.

Image processing module 112 further includes a display module 418 configured to communicate the second volumetric image data set 1100 to a display. Preferably, the second volumetric image data set 1100 has been processed by rendering module 416 for realistic output. Image processing module 112 also includes export module 420 configured to export the second volumetric image data set to a suitable file format such as Digital Imaging and Communications in Medicine (DICOM) file format. Thereby, exchange of the second volumetric image data set between systems may be facilitated.

FIG. 5 illustrates a quadrant view of a first volumetric image data set or volumetric model of a patient's dentition and particularly illustrating spatial relationships between the modeled mandible 506 and modeled maxilla 504 in a closed-bite position. The model comprises three-dimensional digital representations of the teeth 508 of the modeled maxillary (upper) and mandibular (lower) arches in a simulated occlusal position, with opposing teeth 508 shown in overlapping contact to reflect interdigitation of occlusal surfaces. The patient has many metal restorations that significantly reduce the sharpness and data quality of the image data in the region of the teeth, leading to artifacts 502. Additionally, the surfaces of the teeth 508 in the modeled maxilla 504 and the lower arch 506 can overlap in the image data. Segmentation can be used to separate the datasets for the modeled maxilla 504 and the modeled mandible 506, as shown in FIG. 7.

FIG. 5 is divided into four quadrants, as would be presented to a user of diagnostic software common to dental practitioners and radiologists. Each quadrant provides a distinct sectional or perspective view of the modeled dentition, intended to highlight various aspects of occlusion, inter-arch relationships, and diagnostic features relevant to dental assessment and treatment planning. Although FIG. 5 illustrates the volumetric model in a closed-bite position, it should be understood that the image data and volumetric model may be provided in an open-bite position, in another aspect.

In the first quadrant (top-right) of FIG. 5, there is shown a frontal perspective view of the volumetric model, depicting the modeled maxilla 504 and modeled mandible 506 in their natural occlusal arrangement when in a closed-bite position. The anterior modeled teeth 508, including the central and lateral incisors and canines, are shown in overlapping contact and as having artifacts 502 which interfere with clear imaging of the dentition. This view emphasizes the alignment, spacing, and interproximal contacts of the anterior dentition, providing insight into midline alignment, overbite, and overjet conditions.

In the second quadrant (top-left) of FIG. 5, there is shown a horizontal cross-sectional view taken along a plane intersecting the occlusal or bite plane. The section transects modeled teeth of the modeled maxilla 504, capturing a top-down cross-section view of the modeled teeth of the modeled maxilla 504. The view facilitates visualization of the artifacts 502 affecting visualization of the dentition of the patient.

In the third quadrant (bottom-left) of FIG. 5, there is shown a cross-sectional view taken along a vertical plane intersecting the left and right molar regions 510 of both the modeled maxilla 504 and modeled mandible 506. The section reveals the occlusal and buccal-lingual relationships of the molars. This view provides information regarding inter-arch alignment and occlusal surface overlap in the posterior dentition, which is advantageous for evaluating occlusal balance and arch coordination. This view further facilitates visualization of the molars and artifacts 502 affecting their visibility.

In the fourth quadrant (bottom-right) of FIG. 5, there is shown a sagittal sectional view through the anterior region, specifically intersecting the central incisor area of both modeled arches. Artifacts 502 present in this image, such as streaks and distortions, obscure the clarity of the image, particularly with respect to the vertical overlap (overbite) and horizontal overlap (overjet) between the modeled upper and lower incisors. While this view is intended to enable assessment of anterior guidance, incisal edge positioning, and potential discrepancies in anterior tooth angulation, the presence of artifacts 502 make that process challenging.

FIG. 6 illustrates the same quadrant views and sectional orientations as shown in FIG. 5, but with a 3D digital impression 602 of the mandible and the mandibular teeth aligned with the modeled mandible 506 and modeled mandibular teeth, respectively. As previously explained, the 3D digital impression 602 of the patient's dentition may be acquired using a suitable scanning methodology, such as via intra-oral optical scanning. Such a scanning operation produces a 3D digital impression 602 which is a detailed representation of the surface of the patient's dentition. Alignment of the 3D digital impression 602 with the modeled structures is accomplished by alignment module 404 and facilitates clear definition of the boundaries of the modeled structure, as will be explained hereinafter. This is advantageous in image datasets having unclear boundaries, such as that shown in FIG. 4 with artifacts 502 therein.

FIG. 7 illustrates the same quadrant views and sectional orientations as shown in FIG. 5, but with the individual dental structures of the patient modeled mandibular arch segmented by segmentation module 406 and preferably labeled and color-coded for enhanced visibility and identification by a practitioner.

Following segmentation, each modeled maxillary arch, modeled mandibular arch, modeled tooth or molar or other relevant anatomical structure (e.g., crowns, roots, interproximal spaces, occlusal surfaces) is a distinct component of the first volumetric image data set or volumetric model and may be provided a distinct visual representation. This enables selective analysis and visualization of specific regions of interest. The segmentation operation may be based on predefined anatomical landmarks, AI-assisted detection, or practitioner-guided modeling or some other suitable method.

In FIG. 7, segmented modeled components are colored or color-coded for ease of distinction between different components, such as tooth types (e.g., incisors, canines, premolars, molars) or distinct modeled structures (e.g., modeled teeth, maxilla, mandible). Modeled structures may be labeled using an appropriate dental notation to facilitate identification and cross-referencing with diagnostic or treatment records. In other aspects, transparency or isolation views may be provided wherein surrounding anatomy is rendered semi-transparent or hidden, allowing focused inspection of specific teeth, root structures, or occlusal surfaces. Occlusal contact mapping may be shown with visual indicators (e.g., color gradients or force vectors) overlaid on contact points to reflect pressure or alignment at the occlusal interface. In another aspect, spacing between adjacent modeled structures may be highlighted to assist in evaluating tightness of contacts, or the presence of gaps. Also, the visualization elements discussed above may be provided in an overlay format, allowing practitioners to toggle between unsegmented and segmented views, or to superimpose anatomical and diagnostic markers. As shown in FIG. 7, the artifacts 502 may still be present in the first volumetric image data set after segmentation is complete.

FIG. 8 illustrates a unitary volumetric model 802 formed by merging, using modeling module 410, the individual segmented structures of the modeled mandible 506. As illustrated in FIG. 8, modeled tooth 508 surface may appear to be absent in some regions due to metal artifacts changing the internal opacity of the teeth.

In one aspect, the modeled structures of the first volumetric image data set which are not aligned with 3D digital impression 602 are masked or removed from the first volumetric image data set by the masking module 412. Thereby, unitary volumetric model 802 may be viewed in isolation. FIG. 9 illustrates an isolated unitary volumetric model 902 of the modeled mandible 506 with the remaining portions, such as the upper arch, digitally masked.

However, some portions of the upper arch may be retained to include a broad zone around the crowns of the mandibular teeth. The broad zone is included to clean up metal artifact scatter and streaking around the crowns of the mandibular teeth prior to applying corrections based on the 3D digital impression.

FIG. 10 illustrates a shell 1002 formed around the teeth 508 in the unitary volumetric model based on data from the 3D digital impression 602. Image processing module 112 further includes impression module 414 which is configured to adjust the thickness of the 3D digital impression, and preferably thickening the 3D digital impression, to form a shell bounding the at least a portion of the first volumetric image data set and supplementing the boundary of the at least one modeled structure. Shell 1002 bounds or re-encapsulates the at least a portion of the first volumetric image data set and supplements the boundary of the at least one modeled structure.

As seen in FIG. 10, the metal artifacts have resulted in dark or black areas 1004 on the surfaces of modeled tooth 1008 and modeled tooth 1010, respectively. These black areas 1004 are bounded by the shell 1002 created based on teeth data from the 3D digital impression 602. Since shell 1002 is formed by thickening the 3D digital impression 602, the shape and position of the shell 1002 matches the teeth surfaces in the first volumetric image data set.

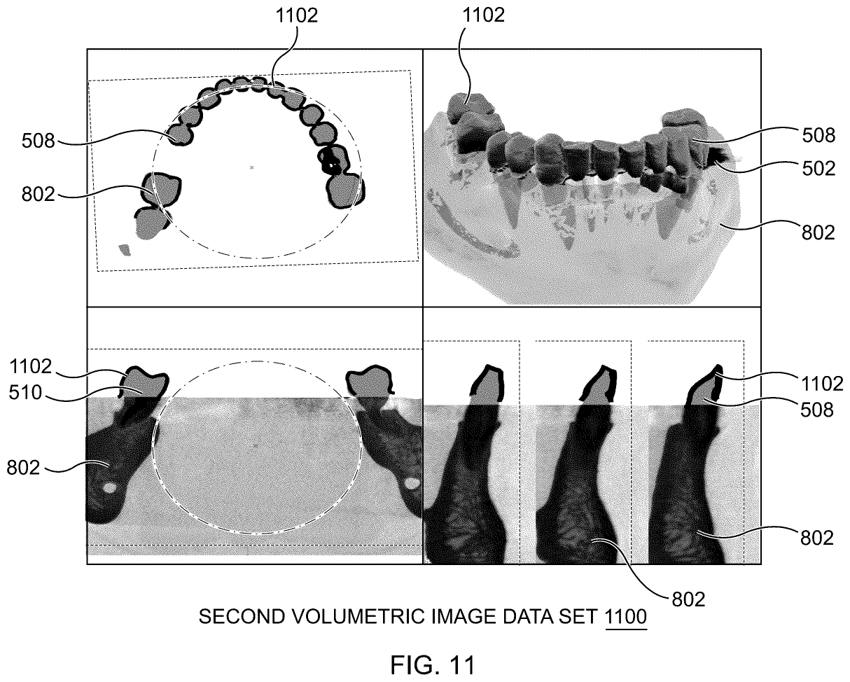

FIG. 11 illustrates the shell 1002 as combined with the first volumetric image data set to produce a well-defined tooth surface for the modeled mandibular arch. Modeling module 410 is configured to combine the 3D digital impression and the unitary volumetric model into a second volumetric image data set 1100 including the unitary volumetric model 802 having at least a portion thereof bounded by the 3D digital impression 602. Thereby a boundary 1102 provided by 3D digital impression 602 is most clearly visible in the section perspective views shown in the second, third and fourth quadrants of FIG. 11.

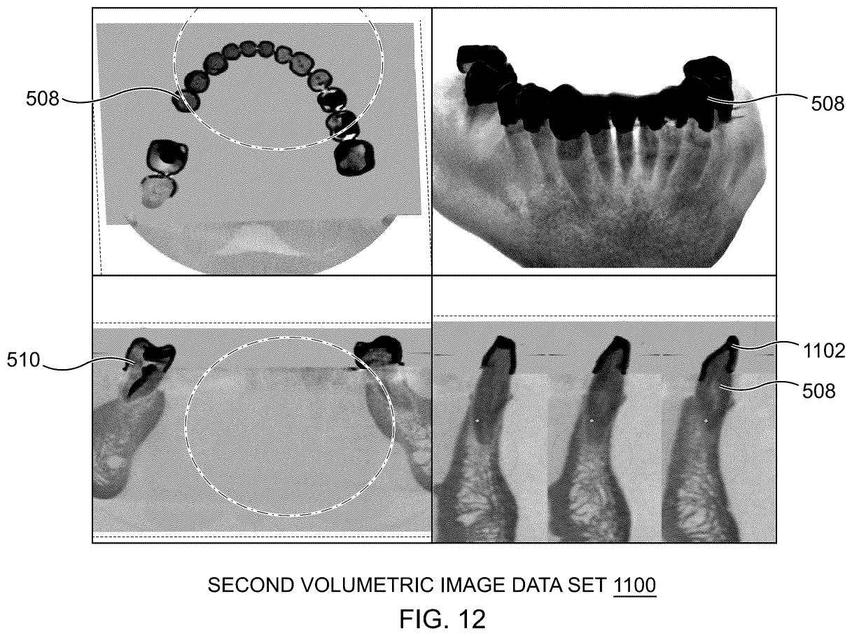

FIG. 12 illustrates the second volumetric image data set 1100 with the modeled mandible 506 and modeled mandibular teeth 508 with surface morphology that matches the 3D digital impression 602. Once the second volumetric image data set 1100 has been generated by the impression module 414, rendering module 416 may render the second volumetric image data set 1100 into a volumetric model having a more realistic visual presentation.

Thereby, there is provided a comprehensive diagnostic perspective of the patient's modeled dentition, facilitating detailed evaluation of occlusal relationships, inter-arch contacts, and morphological features without the presence of streaks or other artifacts which may interfere with clinical use of the volumetric image information. The second volumetric image data set thereby serves as a useful tool for planning dental or orthodontic treatments or for evaluating progress thereof.

While the invention has been described in terms of specific aspects, it is apparent that other forms could be adopted by one skilled in the art. For example, the methods described herein could be performed in a manner which differs from the aspects described herein. The steps of each method could be performed using similar steps or steps producing the same result but which are not necessarily equivalent to the steps described herein. Some steps may also be performed in different order to obtain the same result. Similarly, the apparatuses and systems described herein could differ in appearance and construction from the aspects described herein, the functions of each component of the apparatus could be performed by components of different construction but capable of a similar though not necessarily equivalent function, and appropriate materials could be substituted for those noted. Accordingly, it should be understood that the invention is not limited to the specific aspects described herein. It should also be understood that the phraseology and terminology employed above are for the purpose of disclosing the illustrated aspects, and do not necessarily serve as limitations to the scope of the invention.

What is claimed is:

1. A method comprising:
acquiring a first volumetric image data set representing dentition of a patient, the first volumetric image data set including a modeled structure having an artifact distorting a boundary thereof;
aligning a 3D digital impression of dentition of the patient with at least a portion of the first volumetric image data set;
segmenting individual structures in the first volumetric image data set;
merging the segmented individual structures to form a unitary volumetric model;
thickening the 3D digital impression to form a shell bounding the at least a portion of the first volumetric image data set and supplementing the boundary of the at least one modeled structure; and,
combining the 3D digital impression and the unitary volumetric model into a second volumetric image data set including the unitary volumetric model having at least a portion thereof bounded by the 3D digital impression.

2. The method of claim 1, further comprising:
rendering the second volumetric image data set to provide a volumetric model having realistic appearance.

3. The method of claim 1, wherein the artifact includes a streak extending beyond the boundary and the method further comprises removing the streak.

4. The method of claim 1, wherein the artifact includes a dark region and dark region is bounded by the shell.

5. The method of claim 1, wherein the 3D digital impression is generated using an intra-oral optical scanner.

6. The method of claim 1, wherein thickening the 3D digital impression includes further thickening representations of tooth crowns of the 3D digital impression.

7. The method of claim 1, wherein segmenting further comprises:
segmenting each of a modeled maxilla and modeled mandible of the first volumetric image data set;
providing decoupled volumetric image data representing the modeled maxilla; and,
providing decoupled volumetric image data for the modeled mandible.

8. The method of claim 7 further comprising:
spacing apart the decoupled volumetric image data representing the modeled maxilla and the decoupled volumetric image data representing the modeled mandible.

9. The method of claim 7, wherein the 3D digital impression is aligned with one of the modeled mandible and the modeled maxilla.

10. The method of claim 1, wherein the shell has a uniform thickness.

11. A system comprising:
a capture module configured to acquire a first volumetric image data set representing dentition of a patient, the first volumetric image data set including a modeled structure having an artifact distorting a boundary thereof;
an alignment module configured to align a 3D digital impression of dentition of the patient with at least a portion of the first volumetric image data set;
a segmentation module configured to segment individual structures in the first volumetric image data set;
a modeling module configured to merge the segmented individual structures to form a unitary volumetric model;
an impression module configured to thicken the 3D digital impression to form a shell bounding the at least a portion of the first volumetric image data set and supplementing the boundary of the at least one modeled structure; and,
wherein the modeling module is further configured to combine the 3D digital impression and the unitary volumetric model into a second volumetric image data set including the unitary volumetric model having at least a portion thereof bounded by the 3D digital impression.

12. The system of claim 11, further comprising:
a rendering module configured to render the second volumetric image data set to provide a volumetric model having realistic appearance.

13. The system of claim 11, wherein the artifact includes a streak extending beyond the boundary and the modeling module is further configured to remove the streak.

14. The system of claim 11, wherein the artifact includes a dark region and the modeling module is configured to bound the dark region within the shell.

15. The system of claim 11, wherein the 3D digital impression is generated using an intra-oral optical scanner.

16. The system of claim 11, wherein the impression module is further configured to further thicken representations of tooth crowns of the 3D digital impression.

17. The system of claim 11, wherein the segmentation module is further configured to segment each of a modeled maxilla and modeled mandible of the first volumetric image data set, provide decoupled volumetric image data representing the modeled maxilla and provide decoupled volumetric image data for the modeled mandible.

18. The system of claim 17, wherein the segmentation module is further configured to space apart the decoupled volumetric image data representing the modeled maxilla and the decoupled volumetric image data representing the modeled mandible.

19. The system of claim 17, wherein the alignment module is configured to align the 3D digital impression to one of the modeled mandible and the modeled maxilla.

20. The system of claim 11, wherein the impression module is configured to thicken the shell to a uniform thickness.
